(19) United States
(12) Patent Application Publication
(10) Pub. No.: US 2019/0153525 A1
(43) Pub. Date: May 23, 2019

(54) MULTIPLEXED OPTIMIZED MISMATCH AMPLIFICATION (MOMA)-REAL TIME PCR FOR ASSESSING FETAL WELL BEING

(71) Applicant: The Medical College of Wisconsin, Inc., Milwaukee, WI (US)

(72) Inventors: Aoy Tomita Mitchell, Elm Grove, WI (US); Karl Stamm, Wauwatosa, WI (US)

(73) Assignee: The Medical College of Wisconsin, Inc., Milwaukee, WI (US)

(21) Appl. No.: 16/097,422

(22) PCT Filed: Apr. 29, 2017
(86) PCT No.: PCT/US17/30292

§ 371 Date: Oct. 29, 2018

Related U.S. Application Data
(60) Provisional application No. 62/330,044, filed on Apr. 29, 2016.

(51) Int. Cl. C12Q 1/6858; G16B 20/20

(52) U.S. Cl. CPC C12Q 1/6858; G16B 20/20; C12Q 2600/106; C12Q 2600/112; C12Q 2600/16

(57) ABSTRACT

This invention relates to methods and compositions for assessing an amount of non-native nucleic acids in a sample, such as from a pregnant subject with the non-native nucleic acids being fetal specific. The methods and compositions provided herein can be used to determine risk of a condition, such as a fetal condition, in a pregnant subject.

### RELATED APPLICATIONS

This application claims the benefit under 35 U.S.C. § 119(e) of the filing date of U.S. Provisional Application 62/330,044, filed Apr. 29, 2016, the contents of which are incorporated herein by reference in their entirety.

### FIELD OF THE INVENTION

This invention relates to methods and compositions for assessing an amount of non-native nucleic acids in a sample from a subject. The methods and compositions provided herein can be used to determine risk associated with a fetus. This invention further relates to methods and compositions for assessing the amount of non-native cell-free deoxyribonucleic acid (non-native cell-free DNA, such as fetal-specific cell-free DNA) using multiplexed optimized mismatch amplification (MOMA).

### SUMMARY OF THE INVENTION

The present disclosure is based, at least in part on the surprising discovery that multiplexed optimized mismatch amplification can be used to quantify low frequency non-native nucleic acids in samples from a subject. Multiplexed optimized mismatch amplification embraces the design of primers that can include a 3′ penultimate mismatch for the amplification of a specific sequence but a double mismatch relative to an alternate sequence. Amplification with such primers can permit the quantitative determination of amounts of non-native nucleic acids in a sample, even where the amount of non-native nucleic acids are, for example, below 1%, or even 0.5%, in a heterogeneous population of nucleic acids.

Provided herein are methods, compositions and kits related to such optimized amplification. The methods, compositions or kits can be any one of the methods, compositions or kits, respectively, provided herein, including any one of those of the examples and drawings.

In one aspect, a method of assessing an amount of fetal-specific nucleic acids in a sample from a pregnant subject, the sample comprising fetal-specific nucleic acids. In one embodiment, the method comprises, for each of a plurality of single nucleotide variant (SNV) targets, performing an amplification-based quantitative assay, such as a polymerase chain reaction (PCR) quantification assay, on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and obtaining or providing results from the amplification-based quantitative assays, such as PCR quantification assays, to determine the amount of fetal-specific nucleic acids in the sample. In one embodiment of any one of the methods provided herein, the results are provided in a report.

In one embodiment of any one of the methods provided herein, the method further comprises determining the amount of the fetal-specific nucleic acids in the sample based on the results. In one embodiment of any one of the methods provided herein, the results comprise the amount of the fetal-specific nucleic acids in the sample.

In one aspect, a method of assessing an amount of fetal-specific nucleic acids in a sample from a pregnant subject, the sample comprising fetal-specific and subject nucleic acids is provided. In one embodiment of any one of the methods provided herein, the method comprises obtaining results from an amplification-based quantitative assay, such as a polymerase chain reaction (PCR) quantification assay, for each of a plurality of single nucleotide variant (SNV) targets, performed on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and assessing the amount of fetal-specific nucleic acids based on the results.

In one embodiment of any one of the methods provided herein, the amount of the fetal-specific nucleic acids in the sample is based on the results of the amplification-based quantitative assays, such as PCR quantification assays.

In one embodiment of any one of the methods provided herein, the results are obtained from a report.

In one embodiment of any one of the methods, compositions or kits provided herein, the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

In one embodiment of any one of the methods provided herein, the amount is the ratio or percentage of fetal-specific nucleic acids to total nucleic acids.

In one embodiment of any one of the methods provided herein, the results are informative results of the amplification-based quantitative assays, such as PCR quantification assays. In one embodiment of any one of the methods provided herein, the amount is based on informative results of the amplification-based quantitative assays, such as PCR quantification assays.

In one embodiment of any one of the methods provided herein, the method further comprises selecting informative results of the amplification-based quantitative assays, such as PCR quantification assays. In one embodiment of any one of the methods provided herein, the selected informative results are averaged. In one embodiment of any one of the methods provided herein, the informative results of the amplification-based quantitative assays, such as PCR quantification assays, are selected based on the genotype of the fetal-specific nucleic acids and/or subject nucleic acids (or paternal genotype). In one embodiment of any one of the methods provided herein, the method further comprises obtaining the genotype of the fetal-specific nucleic acids and/or subject nucleic acids (or paternal genotype).

In one embodiment of any one of the methods provided herein, the method further comprises obtaining the plurality of SNV targets. In one embodiment of any one of the methods provided herein, the method further comprises obtaining the at least two primer pairs for each of the plurality of SNV targets. In one embodiment of any one of the methods, compositions or kits provided herein, the plurality of SNV targets is at least 18, 21, 24, 27, 30, 33, 36, 39, 42, 45, 48, 51, 54, 57, 60, 63, 66, 69, 72 or 75 SNV targets. In one embodiment of any one of the methods, compositions or kits provided herein, the plurality of SNV targets is less than 90 SNV targets. In one embodiment of any one of the methods, compositions or kits provided herein, the plurality of SNV targets is less than 75 SNV targets.

In one embodiment of any one of the methods, compositions or kits provided herein, the plurality of SNV targets are informative targets, and the plurality of SNV informative targets is at least 6, 7, 8, 9, 10, 11, 12, 13, 14, 15, 16, 17, 18, 19, 20, 21, 22, 23, 24 or 25 SNV informative targets. In one embodiment of any one of the methods, compositions or kits provided herein, the plurality of SNV informative targets is less than 30 SNV informative targets. In one embodiment of any one of the methods, compositions or kits provided herein, the plurality of SNV informative targets is less than 25 SNV informative targets.

In one embodiment of any one of the methods provided herein, the amount of fetal-specific nucleic acids in the sample is less than 10%. In one embodiment of any one of the methods provided herein, the amount of fetal-specific nucleic acids in the sample is less than 5%.

In one embodiment of any one of the methods provided herein, the paternal genotype is not known or obtained. In one embodiment of any one of the methods provided herein, the method further comprises assessing results based on a prediction of the paternal genotype. In one embodiment of any one of the methods provided herein, the prediction is performed with an expectation-maximization algorithm.

In one embodiment of any one of the methods, compositions or kits provided herein, the fetal-specific nucleic acids are fetal-specific cell-free DNA.

In one embodiment of any one of the methods provided herein, the amount is used to determine a risk to the fetus. In one embodiment of any one of the methods provided herein, the risk to the fetus is the risk of any one of the conditions provided herein.

In one embodiment of any one of the methods, compositions or kits provided herein, the plurality of PCR quantification assays are real time PCR assays or digital PCR assays.

In one embodiment of any one of the methods provided herein, the method further comprises selecting a treatment for the subject based on the amount of fetal-specific nucleic acids. In one embodiment of any one of the methods provided herein, the method further comprises treating the subject based on the amount of fetal-specific nucleic acids. In one embodiment of any one of the methods provided herein, the method further comprises providing information about a treatment to the subject based on the amount of fetal-specific nucleic acids. In one embodiment of any one of the methods provided herein, the method further comprises monitoring or suggesting the monitoring of the amount of fetal-specific nucleic acids in the subject over time.

In one embodiment of any one of the methods provided herein, the method further comprises assessing the amount of fetal-specific nucleic acids in the subject at a subsequent point in time. In one embodiment of any one of the methods provided herein, the method further comprises evaluating an effect of a treatment administered to the subject based on the amount of fetal-specific nucleic acids. In one embodiment of any one of the methods provided herein, the treatment is any one of the treatments provided herein.

In one embodiment of any one of the methods provided herein, the sample is from a subject at at least 10 weeks gestational age. In one embodiment of any one of the methods provided herein, an amount of fetal-specific nucleic acids of less than 10% is indicative of fetal distress, such as at a gestational age of 10 weeks or greater.

In one embodiment of any one of the methods provided herein, the method further comprising providing or obtaining the sample or a portion thereof. In one embodiment of any one of the methods provided herein, the method further comprises extracting nucleic acids from the sample.

In one embodiment of any one of the methods provided herein, the method further comprises a pre-amplification step using primers for the SNV targets. The primers may be the same or different as those for determining the amount of non-native nucleic acids.

In one embodiment of any one of the methods provided herein, the sample comprises blood, plasma or serum.

In one aspect, a composition or kit comprising a primer pair, for each of at least 6 SNV informative targets, wherein each primer pair comprises a 3′ penultimate mismatch relative to one allele of a SNV target but a 3′ double mismatch relative to another allele of the SNV target in a primer and specifically amplifies the one allele of the SNV target is provided. In one embodiment of any one of the compositions or kits provided herein, the composition or kit further comprises another primer pair for each of the at least 6 SNV informative targets wherein the another primer pair specifically amplifies the another allele of the SNV target. In one embodiment of any one of the methods, compositions or kits provided herein, the at least 6 SNV informative targets is at least 7, 8, 9, 10, 11, 12, 13, 14, 15, 16, 17, 18, 19, 20, 21, 22, 23, 24, 25, 26, 27, 28, 29, 30, 31 or 32 SNV informative targets. In one embodiment of any one of the methods, compositions or kits provided herein, the at least 6 SNV informative targets is less than 35 SNV informative targets. In one embodiment of any one of the methods, compositions or kits provided herein, the at least 6 SNV informative targets is less than 30 SNV informative targets. In one embodiment of any one of the methods, compositions or kits provided herein, the at least 6 SNV informative targets is less than 25 SNV informative targets.

In one embodiment of any one of the methods, compositions or kits provided herein, the another primer pair for each of the at least 6 SNV informative targets also comprises a 3′ penultimate mismatch relative to the another allele of the SNV target but a 3′ double mismatch relative to the one allele of the SNV target in a primer and specifically amplifies the another allele of the SNV target.

In one aspect, a composition or kit comprising a primer pair, for each of at least 18 SNV targets, wherein each primer pair comprises a 3′ penultimate mismatch relative to one allele of a SNV target but a 3′ double mismatch relative to another allele of the SNV target in a primer and specifically amplifies the one allele of the SNV target is provided. In one embodiment of any one of the compositions or kits provided herein, the composition or kit further comprises another primer pair for each of the at least 18 SNV targets wherein the another primer pair specifically amplifies the another allele of the SNV target. In one embodiment of any one of the methods, compositions or kits provided herein, the at least 18 SNV targets is at least 21, 24, 27, 30, 33, 36, 39, 42, 45, 48, 51, 54, 57, 60, 63, 66, 69, 71, 75, 78, 81, 84, 87, 90, 93, or 96 SNV targets. In one embodiment of any one of the methods, compositions or kits provided herein, the at least 18 SNV targets is less than 100 SNV targets. In one embodiment of any one of the methods, compositions or kits provided herein, the at least 18 SNV targets is less than 95 SNV targets. In one embodiment of any one of the methods, compositions or kits provided herein, the at least 18 SNV targets is less than 90 SNV targets. In one embodiment of any one of the methods, compositions or kits provided herein, the at least 18 SNV targets is less than 85 SNV targets. In one embodiment of any one of the methods, compositions or kits provided herein, the at least 18 SNV targets is less than 80 SNV targets. In one embodiment of any one of the methods, compositions or kits provided herein, the at least 18 SNV targets is less than 75 SNV targets.

In one embodiment of any one of the methods, compositions or kits provided herein, the another primer pair for each of the at least 18 SNV targets also comprises a 3′ penultimate mismatch relative to the another allele of the SNV target but a 3′ double mismatch relative to the one allele of the SNV target in a primer and specifically amplifies the another allele of the SNV target.

In one embodiment of any one of the compositions or kits provided herein, the composition or kit further comprises a buffer. In one embodiment of any one of the compositions or kits provided herein, the composition or kit further comprises a polymerase. In one embodiment of any one of the compositions or kits provided herein, the composition or kit further comprises a probe. In one embodiment of any one of the compositions or kits provided herein, the probe is a fluorescent probe.

In one embodiment of any one of the compositions or kits provided herein, the composition or kit further comprises instructions for use. In one embodiment of any one of the compositions or kits provided herein, the instructions for use are instructions for determining or assessing the amount of non-native nucleic acids in a sample. In one embodiment of any one of the compositions or kits provided herein, the composition or kit is used in any one of the methods provided herein.

In one aspect, a method comprising obtaining the amount of fetal-specific nucleic acids based any one of the methods provided herein, and assessing a risk in the subject or fetus based on the amount is provided.

In one embodiment of any one of the methods provided, a treatment or information about a treatment or non-treatment is selected for or provided to the subject based on the assessed risk. In one embodiment of any one of the methods provided herein, the method further comprises monitoring or suggesting the monitoring of the amount of fetal-specific nucleic acids in the subject over time. In one embodiment of any one of the methods provided herein, the subject is monitored at one or more points during the gestational period of 10 weeks or greater.

In one aspect, a report containing one or more of the results as provided herein is provided. In one embodiment of any one of the reports provided, the report is in electronic form. In one embodiment of any one of the reports provided, the report is a hard copy. In one embodiment of any one of the reports provided, the report is given orally.

In one embodiment of any one of the methods, compositions or kits provided, the mismatched primer(s) is/are the forward primer(s). In one embodiment of any one of the methods, compositions or kits provided, the reverse primers for the primer pairs for each SNV target is the same.

In one embodiment of any one of the methods provided, the method further comprises obtaining another sample from the subject, such as at a subsequent point in time, and performing a test on the sample, such as any one of the methods provided herein.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

### BRIEF DESCRIPTION OF DRAWINGS

The accompanying drawings are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 provides exemplary amplification traces.

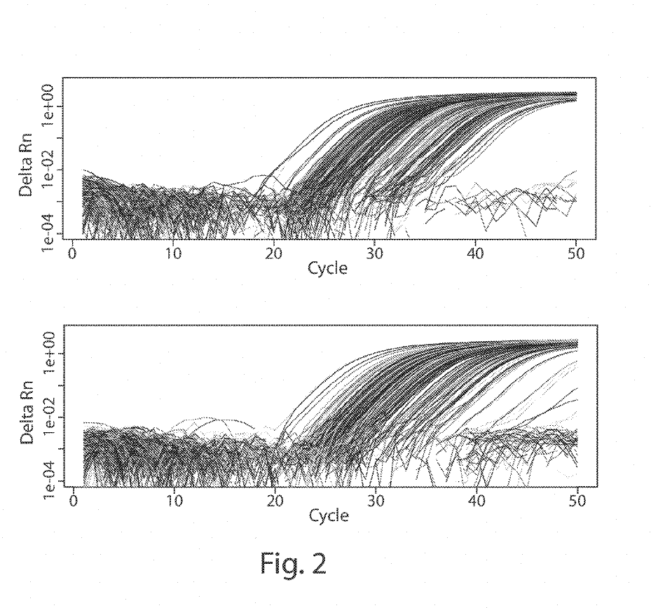

FIG. 3 demonstrates the use expectation maximization to predict non-native genotype when unknown.

FIG. 4 demonstrates the use expectation maximization to predict non-native genotype when unknown.

FIG. 5 provides the average background noise for 104 MOMA targets.

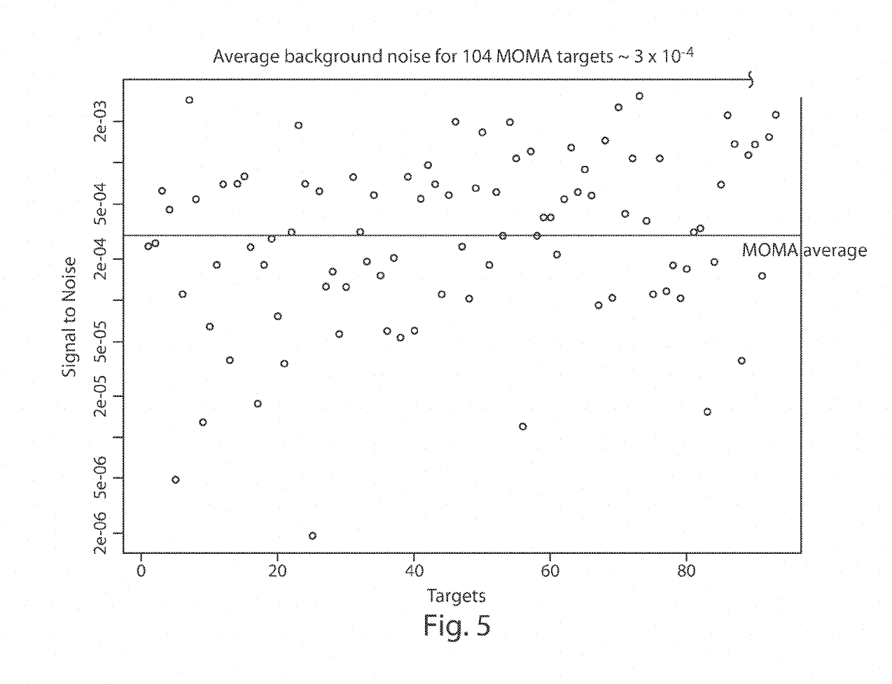

FIG. 6 provides further examples of the background noise for methods using MOMA.

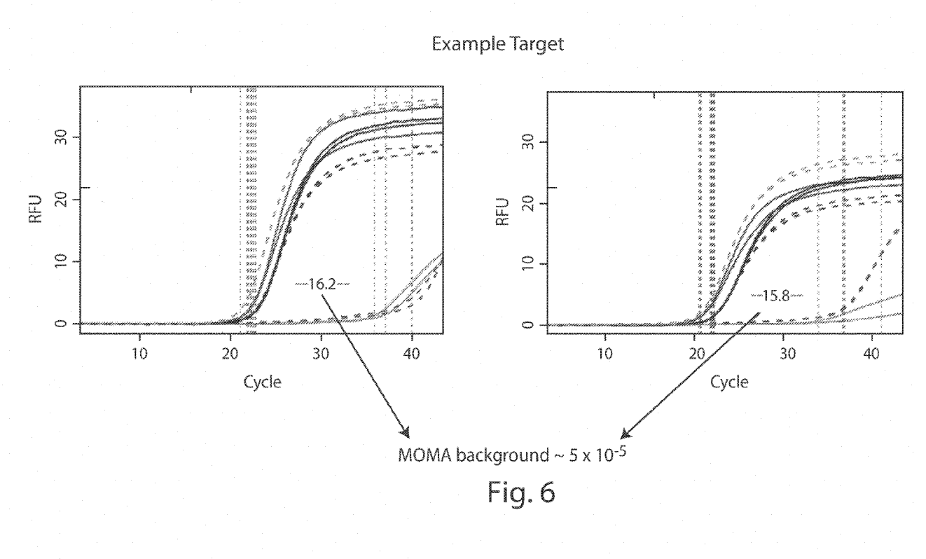

FIG. 7 is a scatterplot showing calculated fetal fraction using a method described herein and fetal gestation age.

FIG. 8 illustrates an example of a computer system with which some embodiments may operate.

### DETAILED DESCRIPTION OF THE INVENTION

Aspects of the disclosure relate to methods for the sensitive detection and/or quantification of non-native nucleic acids in a sample. Non-native nucleic acids, such as non-native DNA, may be present in individuals in a variety of situations including from the fetus of a pregnant subject. The disclosure provides techniques to detect, analyze and/or quantify non-native nucleic acids, such as non-native cell-free DNA concentrations, in samples obtained from a subject.

As used herein, “non-native nucleic acids” refers to nucleic acids that are from another source or are mutated versions of a nucleic acid found in a subject (with respect to a specific sequence). “Native nucleic acids”, therefore, are nucleic acids that are not from another source and are not mutated versions of a nucleic acid found in a subject (with respect to a specific sequence, such as a wild-type (WT) sequence). In some embodiments, the non-native nucleic acid is non-native cell-free DNA. “Cell-free DNA” (or cf-DNA) is DNA that is present outside of a cell, e.g., in the blood, plasma, serum, urine, etc. of a subject. Without wishing to be bound by any particular theory or mechanism, it is believed that cf-DNA is released from cells, e.g., via apoptosis of the cells. An example of non-native nucleic acids are nucleic acids that are from a fetus in a pregnant subject. As used herein, the compositions and methods provided herein can be used to determine an amount of cell-free DNA from a non-native source, such as DNA specific to a fetus or fetal-specific cell-free DNA (e.g., fetal-specific cfDNA).

Provided herein are methods and compositions that can be used to measure nucleic acids with differences in sequence identity. In some embodiments, the difference in sequence identity is a single nucleotide variant (SNV); however, wherever a SNV is referred to herein, any difference in sequence identity between native and non-native nucleic acids is intended to also be applicable. Thus, any one of the methods or compositions provided herein may be applied to native versus non-native nucleic acids where there is a difference in sequence identity. As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. Generally, a “minor allele” refers to an allele that is less frequent, such as in a population, for a locus, while a “major allele” refers to the more frequent allele, such as in a population. The SNVs can be also be known indicia of a fetal condition. In some embodiments of any one of the methods provided herein, such mutations are mutations associated with any one of the conditions provided herein. The methods and compositions provided herein can quantify nucleic acids of major and minor alleles within a mixture of nucleic acids even when present at low levels, in some embodiments. Examples of mutations include mutations associated with any of the fetal conditions provided herein, including high risk gene defects, fetal demise, etc.

The nucleic acid sequence within which there is sequence identity variability, such as a SNV, is generally referred to as a “target”. As used herein, a “SNV target” refers to a nucleic acid sequence within which there is sequence variability, such as at a single nucleotide, such as in a population of individuals or as a result of a mutation that can occur in a subject and that can be associated with a disease or condition. The SNV target has more than one allele, and in preferred embodiments, the SNV target is biallelic. In some embodiments of any one of the methods provided herein, the SNV target is a SNP target. In some of these embodiments, the SNP target is biallelic. It has been discovered that non-native nucleic acids can be quantified even at extremely low levels by performing amplification-based quantitative assays, such as quantitiative PCR assays, with primers specific for SNV targets. In some embodiments, the amount of non-native nucleic acids is determined by attempting amplification-based quantitative assay, such as quantitative PCR, with primers for a plurality of SNV targets.

A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. As used herein, one allele may be the mutated version of a target sequence and another allele is the non-mutated version of the sequence.

In any one of the methods or compositions provided herein the plurality of SNV targets comprises at least 6 informative SNV targets. In some embodiments of any one of the methods or compositions provided, the amplification-based quantitative assay, such as quantitative PCR, is performed with primer pairs for at least 6, 7, 8, 9, 10, 11, 12, 13, 14, 15, 16, 17, 18, 19, 20, 21, 22, 23, 24, 25, 26, 27, 28, 29, 30, 31, or 32 informative targets. In some embodiments of any one of the methods or compositions provided herein, the quantitative assay is performed with primer pairs for fewer than 35, 34, 33, 32, 31, 30, 29, 28, 27, 26, 25, 24, or 23 informative targets. In some embodiments of any one of the methods or compositions provided herein, the quantitative assay is performed with primer pairs for 6-10, 6-15, 6-20, 6-25 or 6-30 informative targets. In some embodiments of any one of the methods or compositions provided herein, the quantitative assay is performed with primer pairs for 7-10, 7-15, 7-20, 7-25 or 7-30 informative targets. In some embodiments of any one of the methods or compositions provided herein, the quantitative assay is performed with primer pairs for 8-10, 8-15, 8-20, 8-25 or 8-30 informative targets. In some embodiments of any one of the methods or compositions provided herein, the quantitative assay is performed with primer pairs for 10-15, 10-20, 10-25 or 10-30 informative targets. In some embodiments of any one of the methods or compositions provided herein, the quantitative assay is performed with primer pairs for 15-20, 15-25 or 15-30 informative targets. In some embodiments of any one of the methods or compositions provided herein, the quantitative assay is performed with primer pairs for 17-20, 17-25 or 17-30 informative targets. In some embodiments of any one of the methods or compositions provided herein, the quantitative assay is performed with primer pairs for 20-25 or 20-30 informative targets.

In an embodiment of any one of the methods or compositions provided herein, primer pairs for SNV targets can be pre-selected based on knowledge that the SNV targets will be informative, such as with knowledge of genotype. In another embodiment of any one of the methods or compositions provided herein, however, primer pairs for SNV targets are selected for the likelihood a percentage will be informative. In such embodiments, primer pairs for a greater number of SNV targets are used based on the probability a percentage of which will be informative. In some embodiments, therefore, of any one of the methods or compositions provided herein informative results are obtained with primer pairs for at least 18, 21, 24, 27, 30, 33, 36, 39, 42, 45, 48, 51, 54, 57, 60, 63, 66, 69, 72, 75, 78, 81, 84, 87, 90, 93 or 96 targets. In some embodiments of any one of the methods or compositions provided herein, the quantitative assay is performed with primer pairs for fewer than 99, 96, 93, 90, 87, 84, 81, 78, 75, 72, or 69 fewer targets. In some embodiments of any one of the methods or compositions provided herein, the quantitative assay is performed with primer pairs for 18-30, 18-45, 18-60, 18-75, 18-80, 18-85, 18-90, 18-95 or 18-100 targets. In some embodiments of any one of the methods or compositions provided herein, the quantitative assay is performed with primer pairs for 21-30, 21-45, 21-60, 21-75, 21-80, 21-85, 21-90, 21-95 or 21-100 targets. In some embodiments of any one of the methods or compositions provided herein, the quantitative assay is performed with primer pairs for 24-30, 24-45, 24-60, 24-75, 24-80, 24-85, 24-90, 24-95 or 24-100 targets. In some embodiments of any one of the methods or compositions provided herein, the quantitative assay is performed with primer pairs for 30-45, 30-60, 30-75, 30-80, 30-85, 30-90, 30-95 or 30-100 targets. In some embodiments of any one of the methods or compositions provided herein, the quantitative assay is performed with primer pairs for 40-45, 40-60, 40-75, 40-80, 40-85, 40-90, 40-95 or 40-100 targets. In some embodiments of any one of the methods or compositions provided herein, the quantitative assay is performed with primer pairs for 45-60, 45-75, 45-80, 45-85, 45-90, 45-95 or 45-100 targets. In some embodiments of any one of the methods or compositions provided herein, the quantitative assay is performed with primer pairs for 50-60, 50-75, 50-80, 50-85, 50-90, 50-95 or 50-100 targets. In some embodiments of any one of the methods or compositions provided herein, an amplification-based quantitative assay, such as quantitative PCR, is performed with primer pairs for at least 40, 45, 50, 55, 60, 65, 70, 75, 80, 85, 90, 91, 92, 93, 94, or 95 targets. In some embodiments of any one of the methods or compositions provided herein, the assay is performed with primer pairs for fewer than 105, 104, 103, 102, 101, 100, 99, 98 or 97 targets. In some embodiments of any one of the methods or compositions provided herein, sufficient informative results are obtained with primer pairs for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93-99, 94-99, 95-99, or 90-95 targets. In some embodiments of any one of the methods or compositions provided herein, sufficient informative results are obtained with primer pairs for between 40-99, 45-99, 50-99, 55-99, 60-99, 65-99, 70-99, 75-99, 80-99, 85-99, 90-99, 95-99, 90-98, 90-97 or 90-96 targets. For any one of the methods or compositions provided, the method or composition can be directed to any one of the foregoing numbers of targets or informative targets.

As used herein, “an informative SNV target” is one in which amplification with primers as provided herein occurs, and the results of which are informative. “Informative results” as provided herein are the results that can be used to quantify the level of non-native or native nucleic acids in a sample. In some embodiments, informative results exclude results where the native nucleic acids are heterozygous for a specific SNV target as well as “no call” or erroneous call results. In the fetal well being-context it should also be recognized that informative non-native nucleic acids can be heterozygous. From the informative results, allele percentages can be calculated using standard curves, in some embodiments of any one of the methods provided. In some embodiments of any one of the methods provided, the amount of non-native and/or native nucleic acids represents an average across informative results for the non-native and/or native nucleic acids, respectively.

The amount or level, such as ratio or percentage, of non-native nucleic acids may be determined with the quantities of the major and minor alleles as well as the genotype of the native and/or non-native nucleic acids. Results can be assessed without knowledge of the genotype of the father. In some embodiments of any one of the methods provided herein, where the genotype of the native nucleic acids is known but the genotype of the father and/or non-native nucleic acids is not known, the method may include a step of predicting the likely non-native genotype or determining the genotype of the father by sequencing. Further details for such methods can be found, for example, in PCT Publication No. WO2016/176662; such methods are incorporated by reference herein. In some embodiments of any one of the methods provided herein, the alleles can be determined based on prior genotyping of the native nucleic acids of the subject and/or the father. Methods for genotyping are well known in the art. Such methods include sequencing, such as next generation, hybridization, microarray, other separation technologies or PCR assays. Any one of the methods provided herein can include steps of obtaining such genotypes.

“Obtaining” as used herein refers to any method by which the respective information or materials can be acquired. Thus, the respective information can be acquired by experimental methods, such as to determine the native genotype. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Reports may be in oral, written (or hard copy) or electronic form, such as in a form that can be visualized or displayed. In some embodiments, the “raw” results for each assay as provided herein are provided in a report, and from this report, further steps can be taken to determine the amount of non-native nucleic acids in the sample. These further steps may include any one or more of the following, selecting informative results, obtaining the native and/or non-native genotype (or father's genotype), calculating allele percentages for informative results for the native and non-native nucleic acids, averaging the allele percentages, etc. In other embodiments, the report provides the amount of non-native nucleic acids in the sample. From the amount, in some embodiments, a clinician may assess the need for a treatment for the subject or the need to monitor the amount of the non-native nucleic acids over time. Accordingly, in any one of the methods provided herein, the method can include assessing the amount of non-nucleic acids in the subject at more than one point in time. Such assessing can be performed with any one of the methods or compositions provided herein.

In some embodiments, any one of the methods provided herein may include a step of determining or obtaining the total amount of nucleic acids, such as total cell-free DNA, in one or more samples from the subject. Accordingly, any one or more of the reports provided herein may also include one or more amounts of the total nucleic acids, such as total cell-free DNA, and the combination of the amount of native and/or non-native nucleic acids that is in a report may be used by a clinician to assess the need for a treatment for the subject or the need to monitor the subject. In some preferred embodiments of any one of the methods provided herein, the total amount of nucleic acids is determined by a MOMA assay as provided herein and is a measure of native and non-native nucleic acid counts as determined by the MOMA assay, preferably, from informative targets. In some embodiments, the total amount of nucleic acids is determined by any method such as a MOMA assay as provided herein or other assays known to those of ordinary skill in the art but not a MOMA assay as provided herein.

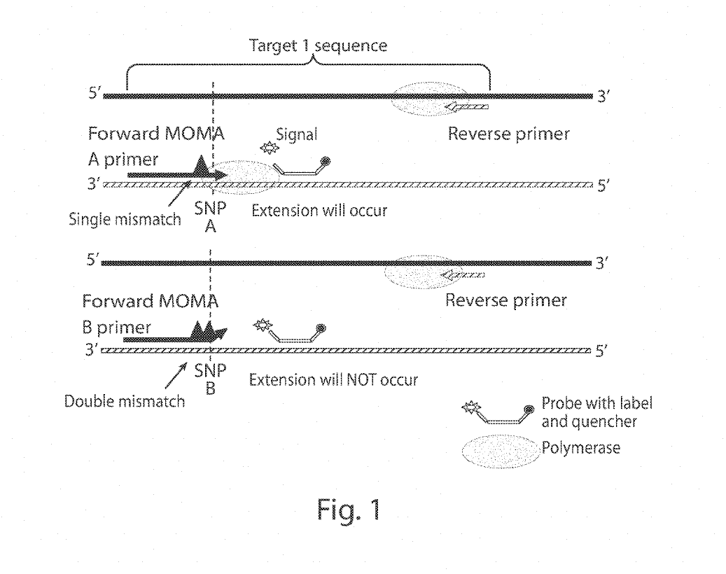

The quantitative assays as provided herein make use of multiplexed optimized mismatch amplification (MOMA). Primers for use in such assays may be obtained, and any one of the methods provided herein can include a step of obtaining one or more primer pairs for performing the amplification-based quantitative assays, such as PCR assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods or compositions provided, this mismatch is at a 3′ nucleotide but adjacent to the SNV position. In some embodiments of any one of the methods or composition provided, the mismatch positioning of the primer relative to a SNV position is as shown in FIG. 1. Generally, such a forward primer even with the 3′ mismatch to produce an amplification product (in conjunction with a suitable reverse primer), such as in a PCR reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods or compositions provided herein, for each SNV target a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods or compositions provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods or compositions provided herein, the mismatch primer is the forward primer. In some embodiments of any one of the methods or compositions provided herein, the reverse primer of the two primer pairs for each SNV target is the same.

These concepts can be used in the design of primer pairs for any one of the compositions and methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

It should be appreciated that the primer pairs described herein may be used in a multiplex amplification-based quantitative assay, such as a PCR assay. Accordingly, in some embodiments of any one of the methods or compositions provided herein, the primer pairs are designed to be compatible with other primer pairs in an amplification reaction, such as a PCR reaction. For example, the primer pairs may be designed to be compatible with at least 2, at least 5, at least 10, at least 20, at least 30, at least 40, etc. other primer pairs in an amplification reaction, such as a PCR reaction. As used herein, primer pairs in an amplification reaction, such as a PCR reaction are “compatible” if they are capable of amplifying their target in the same reaction. In some embodiments, primer pairs are compatible if the primer pairs are inhibited from amplifying their target DNA by no more than 1%, no more than 2%, no more than 5%, no more than 10%, no more than 15%, no more than 20%, no more than 25%, no more than 30%, no more than 35%, no more than 40%, no more than 45%, no more than 50%, or no more than 60% when multiplexed in the same an amplification reaction, such as PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex PCR assay are known in the art or otherwise described herein.

In some embodiments, the primer pairs described herein are used in a multiplex amplification-based assay, such as PCR assay, to quantify an amount of non-native nucleic acids. Accordingly, in some embodiments of any one of the methods or compositions provided herein, the primer pairs are designed to detect genomic regions that are diploid, excluding primer pairs that are designed to detect genomic regions that are potentially non-diploid. In some embodiments of any one of the methods or compositions provided herein, the primer pairs used in accordance with the disclosure do not detect repeat-masked regions, known copy-number variable regions, or other genomic regions that may be non-diploid.

In some embodiments of any one of the methods provided herein, the amplification-based quantitative assay is any quantitative assay, such as whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein, the quantitative assays are quantitative PCR assays. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions of kits provided herein can include one or more probes. Importantly, in some embodiments in any one of the methods provided herein, the probe in one or more or all of the PCR quantification assays is on the same strand as the mismatch primer and not on the opposite strand. It has been found that in so incorporating the probe in a PCR reaction, additional allele specific discrimination can be provided.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

In any one of the methods provided herein, the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication number US20140242582.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrccoccus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction in an amount adequate for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the primers used in accordance with the disclosure are modified. The primers may be designed to bind with high specificity to only their intended target (e.g., a particular SNV) and demonstrate high discrimination against further nucleotide sequence differences. The primers may be modified to have a particular calculated melting temperature (Tm), for example a melting temperature ranging from 46° C. to 64° C. To design primers with desired melting temperatures, the length of the primer may be varied and/or the GC content of the primer may be varied. Typically, increasing the GC content and/or the length of the primer will increase the Tm of the primer. Conversely, decreasing the GC content and/or the length of the primer will typically decrease the Tm of the primer. It should be appreciated that the primers may be modified by intentionally incorporating mismatch(es) with respect to the target in order to detect a particular SNV (or other form of sequence non-identity) over another with high sensitivity. Accordingly, the primers may be modified by incorporating one or more mismatches with respect to the specific sequence (e.g., a specific SNV) that they are designed to bind.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype. The wide range of efficient amplifications shows successful detection of even low concentration nucleic acids.

It has been found that the methods and compositions provided herein can be used to detect low-level nucleic acids, such as non-native nucleic acids like fetal-specific nucleic acids, in a sample. In some embodiments, any one of the methods provided herein can be used on a sample to detect non-native nucleic acids that are at least about 10% in the sample relative to total nucleic acids, such as total cf-DNA (e.g., native plus non-native). In some embodiments, any one of the methods provided herein can be used on a sample to detect non-native nucleic acids that are at least about 7% in the sample. In some embodiments, any one of the methods provided herein can be used on a sample to detect non-native nucleic acids that are at least about 5% in the sample. In some embodiments, any one of the methods provided herein can be used on a sample to detect non-native nucleic acids that are at least about 2.5% in the sample. In some embodiments, any one of the methods provided herein can be used on a sample to detect non-native nucleic acids that are at least about 1% in the sample. In some embodiments, any one of the methods provided herein can be used on a sample to detect non-native nucleic acids that are at least about 0.5% in the sample. In some embodiments, any one of the methods provided herein can be used on a sample to detect non-native nucleic acids that are at least about 0.25% in the sample.

Because of the ability to determine amounts of non-native nucleic acids, even at low levels, the methods and compositions provided herein can be used to assess a risk in a subject, such as a risk to the fetus. A “risk” as provided herein, refers to the presence or absence or progression of any undesirable condition in a subject, or an increased likelihood of the presence or absence or progression of such a condition, such as a fetus or pregnant subject where the condition is adverse to the fetus. The condition can be any one of the conditions provided herein.

In some embodiments the fetus has or is at risk of having badycardia, twin-twin transfusion syndrome (TTTS), gastroschisis, congenital pulmonary airway malformations (CPAMS), hydrops fetalis or fetal arrhythmia. In some embodiments, the fetus has had, or is at risk of having, distress. In some embodiments, the pregnant subject is at risk. In some embodiments, the pregnant subject is at risk for a condition including, for example, chorioamnionitis or preterm labor. From the examples provided herein, it has been demonstrated that levels of fetal specific cf-DNA (cff-DNA), such as the percent or ratio of cff-DNA, can be decreased compared to a baseline whereby the decrease is indicative of an adverse condition of the fetus. Thus, the amounts of fetal specific cf-DNA from a cf-DNA sample obtained from a pregnant subject can provide a sensitive and non-invasive way of monitoring the well being of a fetus and allowing for medical intervention or early delivery, if needed. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or nonprogression) of such a condition. In some embodiments the nucleic acids comprise cf-DNA, which generally comprises DNA of the pregnant subject and DNA of the fetus, where a decreasing amount of the fetal DNA relative to the pregnant subject or total DNA can be indicative of a risk in the fetus and/or indicative of the presence or progression of an adverse condition in the fetus.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values. A determined amount of the fetal specific nucleic acids, such as the percent or ratio of fetal specific cf-DNA, in the sample from the pregnant subject may then be used to determine a risk associated with the fetus. These amounts can be compared relative to a threshold (such as a baseline level) and/or changes in such values can be monitored over time. For example, a change in the difference from a threshold value (such as a baseline) can be used as a non-invasive clinical indicator. This ratio can allow for the measurement of variations in a clinical state and/or permit calculation of normal values or baseline levels. Whatever the form of the amount, in preferred embodiments, the amount can be compared to threshold levels.

An increase or decrease above a threshold (e.g., baseline) in the determined amount, or changes in the increase or decrease over time, can indicate an increased or decreased risk in the fetus. “Threshold”, as used herein, refers to any predetermined level that is indicative of the presence or absence of a condition or the presence or absence of a risk. The threshold value can take a variety of forms. It can be single cut-off value, such as a median or mean. In some embodiments of any one of the methods provided herein, the threshold is any of the medians or means provided herein, such as in the Examples, or that are otherwise known in the art. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. As another example, a threshold values can be a value taken at a prior time point. Such a value can be indicative of a normal or other state in the subject, such as a state not correlated with the risk or condition that is being tested for. Accordingly, the predetermined values selected may take into account the category in which the subject falls. Appropriate ranges and categories can be selected with no more than routine experimentation by those of ordinary skill in the art. In some embodiments, the threshold value can be a baseline value of the subject being tested. In embodiments of any one of the methods provided herein, it is preferred that the comparison is relative to one or more baseline values or changes thereto. Such baseline values can represent an amount of fetal specific nucleic acids, such as the percent or ratio thereof relative to total cf-DNA or non-fetal specific cf-DNA, when the fetus was in good health, prior to the onset of a condition in the fetus or prior to a condition progressing to a deleterious point.

As used herein, a “baseline level” for such subjects can include an amount of fetal-specific nucleic acids, such as specific cf-DNA (such as the percent or ratio of fetal specific cf-DNA relative to the total cf-DNA or non-specific cf-DNA), from a sample from the subject taken at a time prior to a subsequent sample or at a time where the subject (e.g., the pregnant woman or fetus) was or was believed to be in good health, did not have or was not believed to have a condition provided herein, or did have or was believed to have a condition provided herein but the condition was or was believed to be at a stage that did not require treatment or intervention. In any one of the methods provided herein, decrease change relative to the baseline can be an indicator of fetal well-being. In any one of the methods provided herein changes in the difference relative to the baseline at two or more time points can be an indicator of fetal well-being. Accordingly, in any one of the methods provided herein the amount of fetal specific nucleic acids is determined at one or more time points, and the increase or decrease relative to one or more baselines values is determined. In any one of the methods provided herein the amount of fetal specific nucleic acids is determined at two or more time points, and the changes in the difference relative to one or more baseline values is determined. In any of the methods provided herein, the methods can further comprise a step of spiking in an internal standard at known quantities to aid in the quantitation of the fetal-specific nucleic acids.

Any one of the methods provided can further comprise performing another test on the subject, optionally based on the result of a method provided herein or comparison of a result thereof. Such other tests can be any other test known by one of ordinary skill in the art to be useful in determining the presence or absence of a risk, such as to a fetus. Such tests include those that determine other clinical measures of fetal well being, including ultrasound biometrical parameters, fetal heart rate, arterial pressure, amniotic fluid levels, bowel dilation, development of hydrops, resolution of hydrops after intervention and fetal loss. In some embodiments, the pH, PaO2, PaCO2, and/or blood lactate levels are assessed in the fetus and/or the pregnant subject. Another test, in some embodiments, can be performing another test with a method provided herein at one or more additional time points. The methods provided and/or the additional test(s) can be performed at any of a number of time points during pregnancy, for example, time points can include during gestation, during progression of a condition, such as a disease, at fetal intervention, and upon resolution of the condition after intervention. The amount of fetal specific nucleic acids, such as the percent or ratio of fetal specific cf-DNA, may also be determined at any other time during the pregnancy, and may be utilized for short- or long-term surveillance. The determination may be performed instead of or in addition to other tests currently used to assess the condition of a fetus. The ability to detect early risk to a fetus, such as with a non-invasive method, can offer early intervention and better patient outcomes.

Despite decades of research, non-invasive antenatal assessment of fetal well being remains a challenge. There is a need for methods of assessment in pregnancies with antenatal anomalies that have an increased risk of fetal loss. Availability of methods to determine fetal compromise before the development of advanced signs of distress would provide a window of time for the appropriate intervention or early delivery. Indirect and late measurements by methods such as ultrasound or biophysical profile, alerts physicians to problems, such as hydrops fetalis, after they have developed. At this point, too much damage may have been done, and fetal recovery, if possible, is difficult and requires tremendous societal resources. Methods, such as those provided herein, to measure early stages of fetal illness and predict when a fetus is at risk for the development of hydrops or demise before it occurs can be beneficial. The methods provided and related compositions can improve pregnancy outcomes and decrease newborn mortality. While the need is particularly acute in high risk pregnancies, the methods provided can be used as a screening test, such as for preterm labor, in all pregnancies.

Individuals carry non-native DNA sources such as in pregnancy. Fragmented cell free fetal DNA (cff-DNA) originating from the fetus and placental trophoblastic tissue can circulate in maternal plasma at detectable levels from week 5 onward. It has been found that acute changes in fetal well being can result in detectably altered levels of circulating cff-DNA in maternal plasma, particularly as such levels relate to baseline levels. Specifically, it has been found that altered levels of circulating cell free fetal DNA, as compared to baseline levels, are associated with in utero fetal demise, spontaneous abortion, and hydrops fetalis. Thus, changes in levels of circulating cff-DNA can be a viable early indicator of fetal outcome, including the development of preterm labor, hydrops fetalis, and fetal loss, as well as other conditions as provided herein, which ultimately can lead to earlier clinical intervention and improved outcomes. Any one of the methods provided herein can be used to assess the risk of any one of the conditions provided herein.

Generally, the amount and/or changes in the amount (e.g., percent or ratio of fetal specific cf-DNA as provided herein) relative to a threshold, such as a baseline, of fetal specific nucleic acids, such as cf-DNA, can be correlated with fetal distress and related conditions. It is expected, for example, that a decrease in fetal metabolism secondary to hypoxia/ischemia leads to a decrease in maternal cff-DNA levels and precedes the appearance of traditional measures of fetal compromise. It has been demonstrated that altered levels of circulating fetal specific cf-DNA are associated with the development of undesired conditions as provided herein. Quantification of the fractions (or percent or ratio) of fetal specific cf-DNA in maternal serum, therefore, can be performed in order to monitor normal development, decrease preterm labor and allow for timely fetal intervention or planned pre-term delivery before the development of hydrops fetalis, intrauterine death or other adverse conditions and, thus, can provide a window of time for appropriate intervention or early delivery providing a major advance in improving the health and outcome of children.

The methods provided herein, therefore, can be used to identify and/or monitor a variety of fetal conditions. As used herein, a “fetal condition” or “condition of a fetus” refers to any health condition or assessment of well being that may change over time. Such conditions can include the presence or absence of fetal aneuploidy, the gender of the fetus or the Rh blood type of the fetus. In some embodiments of any one of the methods provided herein, the method is not for determining the presence or absence of fetal aneuploidy, the gender of the fetus or the Rh blood type of the fetus. In some embodiments of any one of the methods provided herein, the condition of the fetus is the presence or absence or level of fetal distress (or fetal compromise).

The approach provided herein includes the detection and quantification of nucleic acids, such as cell free DNA, of fetal origin in a maternal sample. It has been found that the fraction (or percent or ratio) of cff-DNA in maternal blood varies as a function of fetal health and can be compared to threshold such as baseline values to assess fetal health. Fetuses can undergo cellular injury during distress from, for example, anatomic, metabolic, or issues of maternal environment that can result in fetal hydrops, a compromised newborn, prematurity or spontaneously terminated pregnancy. Fetal conditions include gastroschisis, fetal cardiac arrhythmias, congenital pulmonary adenomatoid malformations, Semilobar holoprosencephaly, fetal left heart syndrome, congenital defects (neuro, heart and others), low metabolic rate, fetal demise and twin-twin transfusion syndrome. The techniques provided can have broader applicability to detect fetal compromise in still other conditions, such as chorioamnionitis, preterm labor, fetal cardiac arrhythmias (also associated with an increased risk of fetal hydrops and in utero demise), fetal bradycardia (which can occur from a variety of causes, most commonly from maternal collagen vascular diseases, such as Systemic Lupus (SLE)), large congenital pulmonary adenomatoid malformations (CPAMs), etc.

The methods and compositions provided herein, accordingly, can be used in a variety of situations to assess the condition of the fetus. For example, fetal well being can be assessed in a fetus that has been diagnosed with a congenital anomaly, such as congenital heart disease. As another example, fetal well being can be assessed in pregnant women with fetal gastroschisis and fetal bradycardia syndrome. In high or low risk pregnancies, the methods and compositions can allow for the recognition of fetal compromise for early intervention or delivery to prevent fetal loss. The methods and compositions, therefore, can assist in planning a woman's pregnancy and allow for medical intervention, appropriate timing of delivery, etc. Such methods and compositions can allow for the further understanding of pre-term labor and fetal demise and decrease the rate of prematurity and neonatal mortality, which remain unacceptably high. Accordingly, any one of the methods or compositions provided can be for use in any one of the conditions provided herein, such as the foregoing situations or for the foregoing subjects.

The methods provided can be used to monitor any pregnancy, where a fetus has or is suspected of having (or is at risk of) any one of the conditions provided herein. For example, levels or amounts, such as percentages, fractions or ratios, of fetal specific nucleic acids, such as cf-DNA, such as relative to total or non-fetal specific nucleic acids, can be determined using any one of the methods provided herein. The described methods of assessing a risk may be implemented in any suitable manner. For example, the method may be implemented as described below in connection with the Examples and accompanying figures. Changes in cff-DNA can be used to correlate with clinical variables and current non-invasive measures of fetal status, including development of hydrops, resolution of hydrops after intervention, and fetal loss. It should also be appreciated that any one of the methods provided can include a step of correcting the results based on maternal weight and/or gestational age. The methods provided herein, therefore, can be used to identify and/or monitor fetal well-being over several time points during gestation. Such monitoring can also occur after fetal intervention.

Accordingly, the methods provided can be used to monitor low—as well as high-risk pregnancies, including intra-uterine growth restriction and maternal vascular diseases. “High-risk pregnancy” is meant to refer to any pregnancy a clinician would deem at risk for one or more complications or conditions associated with such complication(s). For example, a pregnancy can be considered high-risk when there are potential complications that could affect the mother, the baby, or both. Further examples where a pregnancy may be indicated as high-risk include pregnancies where health problems exist (e.g., diabetes, cancer, high blood pressure, kidney disease (e.g., chronic pyelonephritis, chronic pyelonephritis and renal insufficiency), epilepsy). A pregnancy may also be deemed high risk if the mother uses alcohol or illegal drugs, or smokes; is younger than 17 or older than 35; has a history of multiple pregnancies, has a history of prior miscarriages; or where the fetus is found to have genetic conditions such as Down syndrome, and/or a heart, lung, or kidney problem. High risk pregnancies also include those who had prior or have current problems in pregnancy (e.g., preterm labor, preeclampsia or seizures (eclampsia)); those who have an infection (e.g., HIV, hepatitis C, cytomegalovirus (CMV), chickenpox, rubella, toxoplasmosis, or syphilis); or those taking certain medications (e.g., lithium, phenytoin (such as Dilantin), valproic acid (Depakene), or carbamazepine (such as Tegretol)). Pregnancies where the pregnant woman has certain health problems (e.g., heart valve problems, hypertension, sickle cell disease, asthma, lupus, or rheumatoid arthritis) can also be considered high risk. Other clinical indicators of high-risk pregnancies are known to those of ordinary skill in the art.

In embodiments, any one of the methods provided herein can include the step of providing a therapy (or treatment) or providing information regarding a therapy (or treatment), to the pregnant subject based on any one or more of the comparisons described herein, such as the comparison with a threshold or baseline value, on any one or more of the determined amounts, or change in the amount relative to a threshold or baseline. In still other embodiments, any one of the methods can be used to assess the efficacy of a therapy (or treatment) where improved values can indicate less of a need for the therapy, while worsening values can indicate the need for a therapy, a different therapy, or an increased amount of a therapy. It is expected that the amount (e.g., percentage or ratio) of fetal specific nucleic acids may change with clinical intervention. Any one of the methods provided herein can include the step of evaluating the need or dose of a therapy based on the result of one or more comparisons at one or more time points. In some embodiments, the therapy or intervention involves an in utero intervention (e.g. surgical procedure, administration of a drug) or early delivery. In some embodiments, the information includes written materials containing the information. Written materials can include the written information in electronic form. In some embodiments, the information may be provided as computer-readable instructions. In some embodiments, the information may be provided orally. In any one of the methods provided herein, the method can further comprise recording the administration of a therapy, the providing of information for a therapy or the suggesting of a therapy to the subject. The therapy may be any one of the therapies or treatments provided herein or otherwise known to those of ordinary skill in the art. Alternatively, no change or no therapy or treatment may be determined and suggested based on the amount, and any one of the methods provided herein can include a step of providing such information to the subject.

Aspects of the invention relate to comparing the amount of fetal specific nucleic acids in a sample of a pregnant subject relative to one or more baseline values and, optionally, treating or providing information in regard to a treatment. It may be particularly useful to a clinician to have a report that contains the value(s) provided herein. In one aspect, therefore such reports are provided. In some embodiments, the report provides multiple values for the amounts of fetal-specific nucleic acids with or without total nucleic acids (such as total cell-free DNA) or non-fetal specific nucleic acids. From the amounts, in some embodiments, a clinician may assess the need for a treatment for the subject or the need to monitor the subject over time.

The approaches provided herein can aid in timing intervention and planning delivery of fetuses at risk for loss or development of hydrops and can also aid in monitoring for supportive care to minimize the risk of preterm labor. The ultimate benefit can be a reduction of fetal loss and reduction in infant mortality. Any one of the methods provided herein, therefore, can further include a step of performing or recommending fetal intervention or delivery of the baby before an intrauterine death occurs for the pregnant woman. In some embodiments, the recommending comprises providing information regarding a suggested treatment, such as fetal intervention and/or delivery options to the pregnant woman. In some embodiments of any one of the methods provided herein, the amount of fetal specific nucleic acids in the sample from the pregnant subject may be used to evaluate an effect of a therapy (e.g., positive or negative) on the fetus by correlating (or comparing) a difference or change in the difference in the amount of fetal specific nucleic acids, such as percent or ratio of fetal specific nucleic acids, relative to one or more baseline values. A suitable therapy may be selected based on the correlation or comparison and/or the amount of the therapy administered to the pregnant subject may be increased or decreased. Alternatively, no change or no therapy may be determined based on the correlation or comparison. Choice of therapies and dosing involved with such therapies are within the skill of those in the art.

Any one of the methods provided herein can comprise extracting nucleic acids, such as cell-free DNA, from a sample obtained from a subject, such as a pregnant female. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

As used herein, the sample from a subject can be a biological sample. Examples of such biological samples include whole blood, plasma, serum, urine, etc. In some embodiments, addition of further nucleic acids, e.g., a standard, to the sample can be performed.

In some embodiments of any one of the methods provided herein, a pre-amplification step is performed. An exemplary method of such an amplification is as follows, and such a method can be included in any one of the methods provided herein. Approximately 15 ng of cell free plasma DNA is amplified in a PCR using Q5 DNA polymerase with approximately 100 targets where pooled primers were at 6 uM total. Samples undergo approximately 35 cycles. Reactions are in 25 ul total. After amplification, samples can be cleaned up using several approaches including AMPURE bead cleanup, bead purification, or simply ExoSAP-IT™ it, or Zymo.

The present disclosure also provides methods for determining a plurality of SNV targets for use in any one of the methods provided herein or from which any one of the compositions of primers can be derived. A method of determining a plurality of SNV targets, in some embodiments comprises a) identifying a plurality of highly heterozygous SNVs in a population of individuals, b) designing one or more primers spanning each SNV, c) selecting sufficiently specific primers, d) evaluating multiplexing capabilities of primers, such as at a common melting temperature and/or in a common solution, and e) identifying sequences that are evenly amplified with the primers or a subset thereof.

As used herein, “highly heterozygous SNVs” are those with a minor allele at a sufficiently high percentage in a population. In some embodiments, the minor allele is at least 25%, 26%, 27%, 28%, 29%, 30%, 31%, 32%, 33%, 34% or 35% or more in the population. In any one of these embodiments, the minor allele is less than 50%, 49%, 45%, 44%, 43%, 42%, 41%, or 40% in the population. Such SNVs increase the likelihood of providing a target that is different between the native and non-native nucleic acids.

Primers can be designed to generally span a 70 bp window but some other window may also be selected, such as one between 65 bps and 75 bps, or between 60 bps and 80 bps. Also, generally, it can be desired for the SNV to fall about in the middle of this window. For example, for a 70 bp window, the SNV was between bases 10-60 or 20-50, such as between bases 30-40. The primers as provided herein can be designed to be adjacent to the SNV.

As used herein, “sufficiently specific primers”, were those that demonstrated discrimination between amplification of the intended allele versus amplification of the unintended allele. Thus, with PCR a cycle gap can be desired between amplification of the two. In one embodiment, the cycle gap can be at least a 5, 6, 7 or 8 cycle gap.

Further, sequences can be selected based on melting temperatures, generally those with a melting temperature of between 45-55 degrees C. were selected as “moderate range sequences”. Other temperature ranges may be desired and can be determined by one of ordinary skill in the art. A “moderate range sequence” generally is one that can be amplified in a multiplex amplification format within the temperature. In some embodiments, the GC % content was between 30-70%, such as between 33-66%.

In one embodiment of any one of the methods provided herein, the method can further comprise excluding sequences associated with difficult regions. “Difficult regions” are any regions with content or features that make it difficult to reliably make predictions about a target sequence or are thought to not be suitable for multiplex amplification. Such regions include syndromic regions, low complexity regions, regions with high GC content or that have sequential tandem repeats. Other such features can be determined or are otherwise known to those of ordinary skill in the art.

The present disclosure also provides compositions or kits that can be useful for assessing an amount of non-native nucleic acids in a sample. In some embodiments, the composition or kit comprises a plurality of primer pairs. Each of the primer pairs of the composition or kit can comprise a forward and a reverse primer, wherein there is a 3′ mismatch in one of the primers (e.g., at the penultimate 3′ nucleotide) in some embodiments of any one of the methods, compositions or kits provided herein. In some embodiments of any one of the methods, compositions or kits provided herein, this mismatch is at a 3′ nucleotide and adjacent to the SNV position and when the particular SNV is not present there is a double mismatch with respect to the other allele of the SNV target. In some embodiments of any one of the methods, compositions or kits provided herein, the mismatch primer of a primer pair is the forward primer. In some embodiments of any one of the methods, compositions or kits provided herein, the reverse primer for each allele of a SNV target is the same.

In some embodiments of any one of the methods, compositions or kits there are at least two primer pairs for each of the SNV targets, such as the informative targets. Any one of the methods, compositions or kits provided herein can include at least two primer pairs for each of the SNV targets according to any one of the numbers of SNV targets, including informative targets, as provided herein. In any one of the methods or compositions or kits provided herein the plurality of SNV targets is at least 6 informative SNV targets. In some embodiments of any one of the methods or compositions or kits provided, the plurality of SNV targets is at least 6, 7, 8, 9, 10, 11, 12, 13, 14, 15, 16, 17, 18, 19, 20, 21, 22, 23, 24, 25, 26, 27, 28, 29, 30, 31 or 32 informative targets. In some embodiments of any one of the methods or compositions or kits provided herein, the plurality of SNV targets is fewer than 33, 32, 31, 30, 29, 28, 27, 26, 25, 24 or 23 informative targets. In some embodiments of any one of the methods or compositions or kits provided herein, the plurality of SNV targets is 6-10, 6-15, 6-20, 6-25 or 6-30 informative targets. In some embodiments of any one of the methods or compositions or kits provided herein, the plurality of SNV targets is 7-10, 7-15, 7-20, 7-25 or 7-30 informative targets. In some embodiments of any one of the methods or compositions or kits provided herein, the plurality of SNV targets is 8-10, 8-15, 8-20, 8-25 or 8-30 informative targets. In some embodiments of any one of the methods or compositions or kits provided herein, the plurality of SNV targets is 10-15, 10-20, 10-25 or 10-30 informative targets. In some embodiments of any one of the methods or compositions or kits provided herein, the plurality of SNV targets is 15-20, 15-25 or 15-30 informative targets. In some embodiments of any one of the methods or compositions or kits provided herein, the plurality of SNV targets is 17-20, 17-25 or 17-30 informative targets. In some embodiments of any one of the methods or compositions or kits provided herein, the plurality of SNV targets is 20-25 or 20-30 informative targets.

In an embodiment of any one of the methods or compositions or kits provided herein, the plurality of SNV targets is at least 18, 21, 24, 27, 30, 33, 36, 39, 42, 45, 48, 51, 54, 57, 60, 63, 66, 69, 72, 75, 78, 81, 84, 87, 93, or 96 targets. In some embodiments of any one of the methods or compositions or kits provided herein, the plurality of SNV targets is fewer than 100, 99, 97, 96, 93, 90, 87, 84, 81, 78, 75, 72 or 69 targets. In some embodiments of any one of the methods or compositions or kits provided herein, the plurality of SNV targets is 18-30, 18-45, 18-60, 18-75, 18-80, 18-85, 18-90, 18-95 or 18-100 targets. In some embodiments of any one of the methods or compositions or kits provided herein, the plurality of SNV targets is 21-30, 21-45, 21-60, 21-75, 21-80, 21-85, 21-90, 21-95 or 21-100 targets. In some embodiments of any one of the methods or compositions or kits provided herein, the plurality of SNV targets is 24-30, 24-45, 24-60, 24-75, 24-80, 24-85, 24-90, 24-95 or 24-100 targets. In some embodiments of any one of the methods or compositions or kits provided herein, the plurality of SNV targets is 30-45, 30-60, 30-75, 30-80, 30-85, 30-90, 30-95 or 30-100 targets. In some embodiments of any one of the methods or compositions or kits provided herein, the plurality of SNV targets is 40-45, 40-60, 40-75, 40-80, 40-85, 40-90, 40-95 or 40-100 targets. In some embodiments of any one of the methods or compositions or kits provided herein, the plurality of SNV targets is 45-60, 45-75, 45-80, 45-85, 45-90, 45-95 or 45-100 targets. In some embodiments of any one of the methods or compositions or kits provided herein, the plurality of SNV targets is 50-60, 50-75, 50-80, 50-85, 50-90, 50-95 or 50-100 targets. In some embodiments of any one of the methods, compositions or kits provided, there is a primer pair, such as at least two primer pairs, for at least 40, 45, 50, 55, 60, 65, 70, 75, 80, 85, 90, 91, 92, 93, 94, or 95 targets. In some embodiments of any one of the methods, compositions or kits provided, there is a primer pair, such as at least two primer pairs, for fewer than 105, 104, 103, 102, 101, 100, 99, 98 or 97 targets. In some embodiments of any one of the methods, compositions or kits provided, there is a primer pair, such as at least two primer pairs, for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93, 99, 94-99, 95-99, or 90-95 targets. In some embodiments of any one of the methods, compositions or kits provided, there is a primer pair, such as at least two primer pairs, for between 40-99, 45-99, 50-99, 55-99, 60-99, 65-99, 70-99, 75-99, 80-99, 85-99, 90-99, 90-99, 90-98, 90-97 or 90-96 targets. In some embodiments of any one of the methods, compositions or kits provided, there is a primer pair, such as at least two primer pairs, for between 90-105, 90-104, 90-103, 90-102, 90-101, 90-100, 90-99, 91-99, 92-99, 93, 99, 94-99, 95-99, 90-95 targets. For any one of the methods or compositions or kits provided, the method or composition or kits can be directed to any one of the foregoing numbers of targets or informative targets. Accordingly, any one of the methods or compositions or kits can be directed to at least two primer pairs for any one of the foregoing numbers of targets or informative targets.

In some embodiments of any one of the methods, compositions or kits provided herein, the primer pairs are designed to be compatible for use in an amplification-based quantitative assay, such as a quantitative PCR assay. For example, the primer pairs are designed to prevent primer dimers and/or limit the number of off-target binding sites. It should be appreciated that the plurality of primer pairs of any one of the methods, compositions or kits provided may be optimized or designed in accordance with any one of the methods described herein.

In some embodiments, any one of the compositions or kits provided further comprises a buffer. In some embodiments, the buffers contain additives such as surfactants, dimethyl sulfoxide (DMSO), glycerol, bovine serum albumin (BSA) and polyethylene glycol (PEG) or other PCR reaction additive. In some embodiments, any one of the compositions or kits provided further comprises a polymerase for example, the composition or kit may comprise E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. In some embodiments, any one of the compositions or kits provided further comprises one or more dNTPs (e.g., dATP, dCTP, dGTP, dTTP). In some embodiments, any one of the compositions or kits provided further comprises a probe (e.g., a TAQMAN™ probe).

A “kit,” as used herein, typically defines a package or an assembly including one or more of the compositions of the invention, and/or other compositions associated with the invention, for example, as previously described. Any one of the kits provided herein may further comprise at least one reaction tube, well, chamber, or the like. Any one of the primers, primer systems (such as a set of primers for a plurality of targets) or primer compositions described herein may be provided in the form of a kit or comprised within a kit.

Each of the compositions of the kit may be provided in liquid form (e.g., in solution), in solid form (e.g., a dried powder), etc. A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the methods provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—MOMA Assay with Genotype Information

### SNV Target Selection

Identification of targets for multiplexing in accordance with the disclosure may include one or more of the following steps, as presently described. First, highly heterozygous SNPs are screened on several ethnic control populations (Hardy-Weinberg p>0.25), excluding known difficult regions. Difficult regions can include syndromic regions likely to be abnormal in patients and regions of low complexity, including centromeres and telomeres of chromosomes. Target fragments of desired lengths can then designed in silico. Specifically, as an example, two 20-26 bp primers spanning each SNP's 70 bp window are designed. All candidate primers can then be queried to GCRh37 using BLAST. Those primers that are found to be sufficiently specific can be retained, and monitored for off-target hits, particularly at the 3′ end of the fragment. The off-target candidate hits can be analyzed for pairwise fragment generation that would survive size selection. Selected primers can then be subjected to an in silico multiplexing evaluation. The primers' computed melting temperatures and guanine-cytosine percentages (GC %) can be used to filter for moderate range sequences. An iterated genetic algorithm and simulated annealing can be used to select candidate primers compatible for 400 targets, ultimately resulting in the selection of 800 primers. The 800 primers can be generated and physically tested for multiplex capabilities at a common melting temperature in a common solution. Specifically, primers can be filtered based on even amplification in the multiplex screen and moderate read depth window. Forty-eight assays can be designed for MOMA using the top performing multiplexed SNPs. Each SNP can have a probe designed in WT/MUT at four mismatch choices; eight probes per assay. The new nested primers can be designed within the 70 bp enriched fragments. Finally, the primers can be experimentally amplified with known heterozygous individuals to evaluate amplification efficiency (8 probes×48 assays in triplicate, using TAQMAN™).

A Priori Genotyping Informativeness of Each Assay Using the known mother and father genotypes at each assayed SNP, a subset of informative assays can be selected. If the father's genotype is not known, it can be inferred. Genotypes may also be learned through sequencing, SNP microarray, or application of a MOMA assay on known samples.

### Post Processing Analysis of Multiplex Assay Performance

Patient-specific MOMA probe biases can be estimated across the experimental cohort. Selection iteratively can be refined to make the final fetal-specific percent call.

### Reconstruction Experiment

The sensitivity and precision of the assay was evaluated using reconstructed plasma samples with known mixing ratios.

### Example 2—MOMA Assay with Mother's but not Father's Genotype Information

Targets generally are identified as either non-informative or informative. In fetal, all targets are half-informative when the baby is related to the mother. In a surrogate pregnancy, the targets are half and fully informative targets. Continuing in the context of a related pregnancy, all targets are either non-informative or half. The half-informative show a heterozygous behavior such that a target appearing 5% foreign represents a 10% overall proportion. A non-informative target shows nearly 0% at the background noise level. An expectation maximization (EM) algorithm can be used to infer the most likely assignments for each target, based on the assumption that the informative targets share a common distribution. A distribution such as a “beta” distribution can be used to give a continuous probability distribution between 0 and 1. On the assumption that all targets are either the background-noise distribution or the fetal-specific proportion, an EM algorithm fills in the unknown target categorization.

For example, consider a set of ten targets, each with a called fetal-specific percentage. The first guess is that any targets calling less than the 25th percentile are background noise and the others are half-informative. With the first guess in place, the EM may begin. Next, the maximization step is performed. A distributional model, such as a beta distributional model is fit over each point group, minimizing the sum of the log-likelihood function of the data points to a two-dimensional model. The beta distribution is defined by two shape parameters. In total, four shape parameters are collected (two per target informativity group). The maximum likelihood estimate of the non-native % is equal to twice the mean of the beta model of the half-informative group. In the subsequent expectation step, all of the targets are evaluated to find to which group each has a higher likelihood of belonging to. Targets are reassigned to non or half-informative groups based on which model has the higher likelihood. If any targets have changed groups, the maximization step is repeated, followed by the expectation step again. Upon convergence, the EM is complete and reports the maximum likelihood informativity categorization of all targets and the final model parameters.

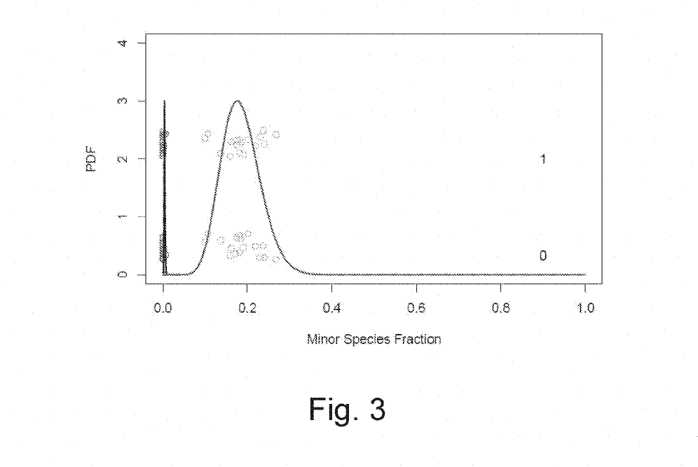

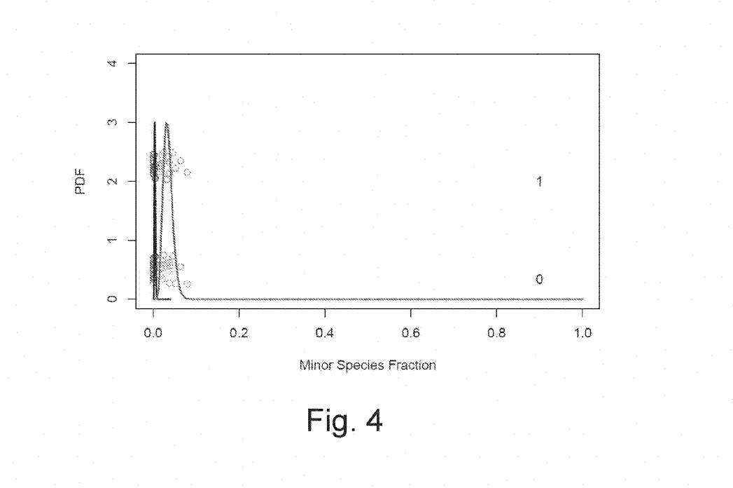

Examples of inferring a genotype are provided in FIGS. 3 and 4, where EM was used to determine fetal specific calculations where the father's genotype was not known. The method was able to distinguish non-informative from half-informative sites. These are then multiplied by 2 to determine the fetal fraction.

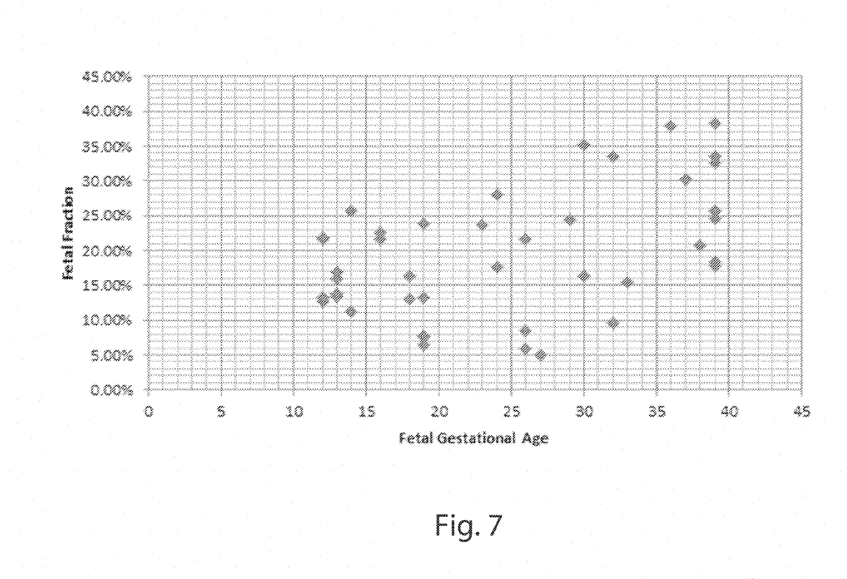

In addition, it has been found that the methods provided herein provide results that correlate fetal fraction with fetal well being. For example, using a method provided herein a low fetal fraction correlated with a fetal demise subject. It was also found that a fetal fraction of 1-10% after fetal gestational age of 10 week indicated developmental pathology. FIG. 7 provides a scatterplot illustrating the fetal fraction of different samples. Maternal samples were taken at the week of gestation indicated (weeks 12-39), and the fetal fraction was calculated inferring the father's genotype, using a method described herein. The percent fetal fraction was then compared with ultrasound results, and fetal fractions 10% or lower after fetal gestational age of 10 weeks were found to indicate developmental pathology.

### Example 3—MOMA Cf-DNA Assay

### Principles and Procedures of a MOMA Cf-DNA Assay

This exemplary assay is designed to determine the percentage of non-native cf-DNA present in a transplant recipient's blood sample. In this embodiment, the maternal blood sample is collected in an EDTA tube and centrifuged to separate the plasma and buffy coat. The plasma and buffy coat can be aliquoted into two separate 15 mL conical tubes and frozen. The plasma sample can be used for quantitative genotyping (qGT), while the buffy coat can be used for basic genotyping (bGT) of the maternal sample.

The first step in the process can be to extract cell free DNA from the plasma sample (used for qGT) and genomic DNA (gDNA) from the buffy coat, whole blood, or tissue sample (used for bGT). The total amount of cfDNA can be determined by qPCR and normalized to a target concentration. This process is known as a cfDNA Quantification. gDNA can be quantified using UV-spectrophotometry and normalized. Fifteen ng of DNA generally provides accurate and valid results.

The normalized patient DNA can be used as an input into a highly-multiplexed library PCR amplification reaction containing, for example, 96 primer pairs, each of which amplify a region including one of the MOMA target sites. The resulting library can be used as the input for either the bGT or qGT assay as it consists of PCR amplicons having the MOMA target primer and probe sites. This step can improve the sensitivity of the overall assay by increasing the copy number of each target prior to the highly-specific qPCR amplification. Controls and calibrators/standards can be amplified with the multiplex library alongside subject samples. Following the library amplification, an enzymatic cleanup can be performed to remove excess primers and unincorporated deoxynucleotide triphosphates (dNTPs) to prevent interference with the downstream amplification.

In a parallel workflow the master mixes can be prepared and transferred to a 384-well PCR plate. The amplified samples, controls, and calibrators/standards can then be diluted with the library dilution buffer to a predetermined volume and concentration. The diluted samples and controls can be aliquoted to a 6-well reservoir plate and transferred to the 384-well PCR plate using an acoustic liquid handler. The plate can then be sealed and moved to a real-time PCR amplification and detection system.

MOMA can perform both the basic and quantitative genotyping analyses by targeting SNVs, such as biallelic SNPs, that can be informative. The basic genotyping analysis can label the three possible genotypes at each target (e.g. homozygous REF, heterozygous REF and VAR, and homozygous VAR). This information can be used for the quantitative genotyping analysis, along with standard curves, to quantitate to the allele ratio for each target, known as a minor-species proportion. The median of all informative and quality-control passed allele ratios can be used to determine the % of fetal-specific cfDNA.

### Example 4—Examples of Computer-Implemented Embodiments

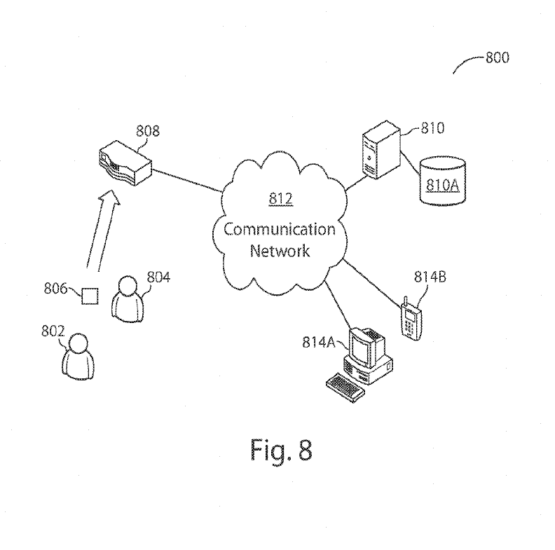

In some embodiments, the diagnostic techniques described above may be implemented via one or more computing devices executing one or more software facilities to analyze samples for a subject over time, measure nucleic acids (such as cell-free DNA) in the samples, and produce a diagnostic result based on one or more of the samples. FIG. 8 illustrates an example of a computer system with which some embodiments may operate, though it should be appreciated that embodiments are not limited to operating with a system of the type illustrated in FIG. 8.

The computer system of FIG. 8 includes a subject 802 and a clinician 804 that may obtain a sample 806 from the subject 802. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 806 so as to determine (including estimate) a total amount of nucleic acids (such as cell-free DNA) and an amount of a non-native nucleic acids (such as cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time to one or more thresholds. For example, the diagnostic facility may determine a risk to the subject 802 of a condition by comparing an amount of nucleic acids (such as cell-free DNA) for one or more samples 806 to a threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

What is claimed is:

1. A method of assessing an amount of fetal-specific nucleic acids in a sample from a pregnant subject, the sample comprising fetal-specific and subject nucleic acids, the method comprising:
for each of a plurality of single nucleotide variant (SNV) targets, performing an amplification-based quantitative assay, such as a polymerase chain reaction (PCR) quantification assay, on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target,
and obtaining or providing results from the amplification-based quantitative assays, such as PCR quantification assays, to determine the amount of fetal-specific nucleic acids in the sample.

2. The method of claim 1, wherein the results are provided in a report.

3. The method of claim 1 or 2, wherein the method further comprises determining the amount of the fetal-specific nucleic acids in the sample based on the results.

4. The method of claim 1 or 2, wherein the results comprise the amount of the fetal-specific nucleic acids in the sample.

5. A method of assessing an amount of fetal-specific nucleic acids in a sample from a pregnant subject, the sample comprising fetal-specific and subject nucleic acids, the method comprising:
obtaining results from an amplification-based quantitative assay, such as a polymerase chain reaction (PCR) quantification assay, for each of a plurality of single nucleotide variant (SNV) targets, performed on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and
assessing the amount of fetal-specific nucleic acids based on the results.

6. The method of claim 5, wherein the amount of the fetal-specific nucleic acids in the sample is based on the results of the amplification-based quantitative assays, such as PCR quantification assays.

7. The method of claim 5 or 6, wherein the results are obtained from a report.

8. The method of any one of the preceding claims, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

9. The method of any one of the preceding claims, wherein the amount is the ratio or percentage of fetal-specific nucleic acids to total nucleic acids.

10. The method of any one of the preceding claims, wherein the results are informative results of the amplification-based quantitative assays, such as PCR quantification assays.

11. The method of any one of the preceding claims, wherein the amount is based on informative results of the amplification-based quantitative assays, such as PCR quantification assays.

12. The method of any one of the preceding claims, wherein the method further comprises selecting informative results of the amplification-based quantitative assays, such as PCR quantification assays.

13. The method of claim 12, wherein the selected informative results are averaged.

14. The method of claim 12 or 13, wherein the informative results of the amplification-based quantitative assays, such as PCR quantification assays, are selected based on the genotype of the fetal-specific nucleic acids and/or subject nucleic acids (or paternal genotype).

15. The method of any one of the preceding claims, wherein the method further comprises obtaining the genotype of the fetal-specific nucleic acids and/or subject nucleic acids (or paternal genotype).

16. The method of any one of the preceding claims, wherein the method further comprises obtaining the plurality of SNV targets.

17. The method of any one of the preceding claims, wherein the method further comprises obtaining the at least two primer pairs for each of the plurality of SNV targets.

18. The method of any one of the preceding claims, wherein the plurality of SNV targets is at least 18, 21, 24, 27, 30, 33, 36, 39, 42, 45, 48, 51, 54, 57, 60, 63, 66, 69, 72, 75, 78, 81, 84, 87, 90, 93 or 96 SNV targets.

19. The method of claim 18, wherein the plurality of SNV targets is less than 100, 99, 98, 97, 96, 95, 94, 93, 92, 91 or 90 SNV targets.

20. The method of claim 19, wherein the plurality of SNV targets is less than 75 SNV targets.

21. The method of any one of claims 1-17, wherein the plurality of SNV targets are informative targets, and the plurality of SNV informative targets is at least 6, 7, 8, 9, 10, 11, 12, 13, 14, 15, 16, 17, 18, 19, 20, 21, 22, 23, 24, 25, 26, 27, 28, 29, 30, 31 or 32 SNV informative targets.

22. The method of claim 21, wherein the plurality of SNV informative targets is less than 34, 33, 32, 31 or 30 SNV informative targets.

23. The method of claim 22, wherein the plurality of SNV informative targets is less than 25 SNV informative targets.

24. The method of any one of the preceding claims, wherein the amount of fetal-specific nucleic acids in the sample is less than 10%.

25. The method of claim 24, wherein the amount of fetal-specific nucleic acids in the sample is less than 5%.

26. The method of any one of the preceding claims, wherein when the paternal genotype is not known or obtained, the method further comprises:
assessing results based on a prediction of the paternal genotype.

27. The method of claim 26, wherein the assessing is performed with an expectation-maximization algorithm.

28. The method of any one of the preceding claims, wherein the fetal-specific nucleic acids are fetal-specific cell-free DNA.

29. The method of claim 28, wherein the amount is used to determine a risk to the fetus.

30. The method of claim 29, wherein the risk to the fetus is the risk of any one of the conditions provided herein.

31. The method of any one of the preceding claims, wherein the plurality of PCR quantification assays are real time PCR assays or digital PCR assays.

32. The method of any one of the preceding claims, wherein the method further comprises selecting a treatment for the subject based on the amount of fetal-specific nucleic acids.

33. The method of any one of the preceding claims, wherein the method further comprises treating the subject based on the amount of fetal-specific nucleic acids.

34. The method of any one of the preceding claims, wherein the method further comprises providing information about a treatment to the subject based on the amount of fetal-specific nucleic acids.

35. The method of any one of the preceding claims, wherein the method further comprises monitoring or suggesting the monitoring of the amount of fetal-specific nucleic acids in the subject over time.

36. The method of any one of the preceding claims, wherein the method further comprises assessing the amount of fetal-specific nucleic acids in the subject at a subsequent point in time.

37. The method of any one of the preceding claims, wherein the method further comprises evaluating an effect of a treatment administered to the subject based on the amount of fetal-specific nucleic acids.

38. The method of any one of claims 32-37, wherein the treatment is any one of the treatments provided herein.

39. The method of any one of the preceding claims, wherein the sample is from a subject at at least 10 weeks gestational age.

40. The method of claim 39, wherein an amount of fetal-specific nucleic acids of less than 10% is indicative of fetal distress.

41. The method of any one of the preceding claims, further comprising providing or obtaining the sample or a portion thereof.

42. The method of any one of the preceding claims, further comprising extracting nucleic acids from the sample.

43. The method of any one of the preceding claims, the method further comprises a pre-amplification step using primers for the SNV targets.

44. The method of any one of the preceding claims, wherein the sample comprises blood, plasma or serum.

45. A composition or kit comprising,
a primer pair, for each of at least 6 SNV informative targets, wherein each primer pair comprises a 3′ penultimate mismatch relative to one allele of a SNV target but a 3′ double mismatch relative to another allele of the SNV target in a primer and specifically amplifies the one allele of the SNV target.

46. The composition or kit of claim 45, further comprising another primer pair for each of the at least 6 SNV informative targets wherein the another primer pair specifically amplifies the another allele of the SNV target.

47. The composition or kit of claim 45 or 46, wherein the at least 6 SNV informative targets is at least 7, 8, 9, 10, 11, 12, 13, 14, 15, 16, 17, 18, 19, 20, 21, 22, 23, 24, 25, 26, 27, 28, 29, 30, 31 or 32 SNV informative targets.

48. The composition or kit of claim 47, wherein the at least 6 SNV informative targets is less than 35, 34, 33, 32, 31, 30, 29, 28, 27, 26 or 25 SNV informative targets.

49. The composition or kit of any one of claims 45-48, wherein the another primer pair for each of the at least 6 SNV informative targets also comprises a 3′ penultimate mismatch relative to the another allele of the SNV target but a 3′ double mismatch relative to the one allele of the SNV target in a primer and specifically amplifies the another allele of the SNV target.

50. A composition or kit comprising,
a primer pair, for each of at least 18 SNV targets, wherein each primer pair comprises a 3′ penultimate mismatch relative to one allele of a SNV target but a 3′ double mismatch relative to another allele of the SNV target in a primer and specifically amplifies the one allele of the SNV target.

51. The composition or kit of claim 50, further comprising another primer pair for each of the at least 18 SNV targets wherein the another primer pair specifically amplifies the another allele of the SNV target.

52. The composition or kit of claim 50 or 51, wherein the at least 18 SNV targets is at least 21, 24, 27, 30, 33, 36, 39, 42, 45, 48, 51, 54, 57, 60, 63, 66, 69, 71, 75, 80, 85, 90, or 95 SNV targets.

53. The composition or kit of claim 52, wherein the at least 18 SNV targets is less than 100, 99, 98, 97, 96, 95, 94, 93, 92, 91 or 90 SNV targets.

54. The composition or kit of claim 53, wherein the at least 18 SNV targets is less than 75 SNV targets.

55. The composition or kit of any one of claims 50-54, wherein the another primer pair for each of the at least 18 SNV targets also comprises a 3′ penultimate mismatch relative to the another allele of the SNV target but a 3′ double mismatch relative to the one allele of the SNV target in a primer and specifically amplifies the another allele of the SNV target.

56. The composition or kit of any one of claims 45-55, further comprising a buffer.

57. The composition or kit of any one of claims 45-56, further comprising a polymerase.

58. The composition or kit of any one of claims 45-57, further comprising a probe.

59. The composition or kit of claim 58, wherein the probe is a fluorescent probe.

60. The composition or kit of any one of claims 45-59, further comprising instructions for use.

61. The composition or kit of claim 60, wherein the instructions for use are instructions for determining or assessing the amount of non-native nucleic acids in a sample.

62. The composition or kit of any one of claims 45-61 for use in a method of any one of claims 1-43.

63. The composition or kit of any one of claims 45-61, for use in any one of the methods provided herein.

64. A method comprising:
obtaining the amount of fetal-specific nucleic acids based on the method of any one of claims 1-44, and
assessing a risk in the subject or fetus based on the amount.

65. The method of claim 64, wherein a treatment or information about a treatment or non-treatment is selected for or provided to the subject based on the assessed risk.

66. The method of claim 64 or 65, wherein the method further comprises monitoring or suggesting the monitoring of the amount of fetal-specific nucleic acids in the subject over time.

67. The method of claim 66, wherein the subject is monitored at one or more points during the gestational period of 10 weeks or greater.
